(19) United States
(12) Patent Application Publication
(10) Pub. No.: US 2016/0089103 A1
(43) Pub. Date: Mar. 31, 2016

(54) SYSTEMS AND METHODS FOR OPTIMIZED IMAGE ACQUISITION WITH IMAGE-GUIDED DECISION SUPPORT

(71) Applicant: General Electric Company, Schenectady, NY (US)

(72) Inventors: Robert David Darrow, Scotia, NY (US); Thomas Kwoh-Fah Foo, Niskayuna, NY (US)

(21) Appl. No.: 14/499,353

(22) Filed: Sep. 29, 2014

(51) Int. Cl. A61B 6/00; A61B 5/055; A61B 5/00; A61B 6/03

(52) U.S. Cl. CPC A61B 6/545; A61B 6/032; A61B 5/055; A61B 5/0037; A61B 5/0059; A61B 6/037

(57) ABSTRACT

A system for optimized image acquisition includes an user interface for receiving a first input indicating a selection of an anatomical region of a subject and receiving a second input indicating a selection of at least one feature of interest and at least one appropriate property of the at least one feature of interest. Further, the system includes a scanning unit for moving the subject to isocenter of a magnet and acquiring at least one image of the anatomical region of the subject. Also, the system includes a processor for processing the at least one image for identifying the at least one feature of interest in the at least one image, wherein the scanning unit is configured to re-scan the at least one feature of interest using the at least one selected appropriate property for acquiring an optimized image of the at least one feature of interest.

### BACKGROUND

The disclosure relates generally to medical imaging and more specifically to systems and methods for optimized image acquisition with image-guided decision support.

In the field of medical imaging, certain regions of patient's anatomy may be of particular interest to a clinician. Particularly, the clinician may have to assess whether one or more organs/regions in the patient's anatomy are healthy or diseased. To that end, the clinicians may use imaging systems to obtain medical images of the anatomy and may inspect the region of interest in the anatomy. In addition, the clinicians may have to perform a number of tasks during the acquisition of medical images and subsequently manipulate the resulting images in order to better assess these organs or regions of interest for making accurate clinical diagnosis. These tasks may include visual inspection of images to identify anomalies, or image processing of already acquired images to generate parametric maps of different anatomical properties to better delineate diseased tissue.

Conventional medical imaging systems are strictly imaging and viewing devices, providing little analysis or recommendations for patient follow-up. For example, MRI exams are typically conducted by skilled operators or clinicians. These operators may have a basic knowledge of patient's anatomy and the pulse sequences required for a given type of clinical examination. However, there is a range of other factors that determine the quality of MRI images.

Also, depending on the MRI imaging center, a particular type of examination may not be frequently performed. For example, if the majority of the examinations are performed for head or abdominal, cardiac MR examinations may be infrequent. As such, the scan operator may not have much knowledge for scanning the heart to produce optimized images for such specific examinations. For example, when scanning a volume of interest (VOI) within patient's anatomy, there are varied tissues present in the volume, each tissue with different characteristics. These varied tissues may in turn make it difficult to image the VOI to generate an optimized image that has correct image contrast and correct imaging parameters for generating motion-free images or images of sufficient spatial or temporal resolution. If the operator selects a fixed protocol, this may not be necessarily ideal for a particular patient or the specific examination. Particularly, in cardiac MRI examination, the ability of the patient to maintain a breath-hold is essential to generate motion-free images. If the operator selects a pre-determined or “canned” protocol, it may have a scan time that is beyond the breath-hold capacity of a particular patient. As a result, the images acquired will be compromised by motion-related artifacts, which in turn degrade the image quality to render them non-diagnostic. Further, the operator is faced with decisions to use either multiple signal averages or modifying the scan parameters to reduce the scan time to within the patient's capacity. In addition, such decisions would be difficult if these types of scans are performed infrequently.

Thus, there is a need for a medical imaging system that automatically provides optimized images of desired regions or features of interest within a patient's anatomy. The optimized images may include processed or computed parametric images. Also, there is a need for an image acquisition and viewing device that can provide future workflow guidance for a patient based on the analysis of selected features.

### BRIEF DESCRIPTION

In accordance with one embodiment described herein, a method includes receiving a first input indicating a selection of an anatomical region of a subject. Also, the method includes receiving a second input indicating a selection of at least one feature of interest and at least one appropriate property of the at least one feature of interest. Further, the method includes moving the subject to isocenter of a magnet. In addition, the method includes automatically scanning the anatomical region of the subject for acquiring at least one image of the anatomical region of the subject. Furthermore, the method includes processing the at least one image for identifying the anatomy scanned and the different tissue organs in the initial imaging field-of-view. Thus, this identifies at least one feature of interest in the at least one image. Also, the method includes automatically re-scanning the at least one feature of interest using the at least one appropriate property for acquiring an optimized image of the at least one feature of interest. The re-scanning and application of the at least one appropriate property includes moving the patient so that the targeted feature of interest is fully in the imaging volume, and/or modifying the acquisition parameters to generate an image of the at least one feature of interest that has the desired image contrast, i.e., an optimized image.

In accordance with a further aspect of the present disclosure, a system includes an user interface configured to receive a first input indicating a selection of an anatomical region of a subject and receive a second input indicating a selection of at least one feature of interest and at least one appropriate property of the at least one feature of interest. Further, the system includes a scanning unit coupled to the user interface and configured to move the subject to isocenter of a magnet to landmark the anatomical region of the subject and acquiring at least one image of the anatomical region of the subject. Also, the system includes a processor coupled to the scanning unit and configured to process the at least one image for identifying the at least one feature of interest in the at least one image, wherein the scanning unit is configured to re-scan the at least one feature of interest using the at least one selected appropriate property (including acquisition parameters to generate the desired image contrast) for acquiring an optimized image of the at least one feature of interest.

### DRAWINGS

These and other features, and aspects of embodiments of the present technique will become better understood when the following detailed description is read with reference to the accompanying drawings in which like characters represent like parts throughout the drawings, wherein:

FIG. 1 is a pictorial view of an exemplary medical imaging system, in accordance with aspects of the present disclosure;

FIG. 2 is a flowchart depicting an exemplary method for acquiring an optimized image of an object, in accordance with aspects of the present disclosure;

FIG. 3 is an image of an anatomical region of the subject with a representative image acquisition plane, in accordance with aspects of the present disclosure;

FIG. 4 is an image of the same anatomical region of the subject, but at a different time, with a representative image acquisition plane, in accordance with aspects of the present disclosure;

FIG. 5 is a set of images representing a correct image acquisition plane, in accordance with aspects of the present disclosure;

FIG. 6 is a set of pre-stored or earlier acquired images of the anatomical region of the subject; and

FIG. 7 is a diagrammatical representation of the image showing a computed parametric map of tissue over-laid on an earlier acquired image that provides anatomical information, in accordance with aspects of the present disclosure.

### DETAILED DESCRIPTION

As will be described in detail hereinafter, various embodiments of exemplary systems and methods for optimized image acquisition with image-guided decision support are presented. By employing the methods and the various embodiments of the system described hereinafter, optimized images of selected features for specific properties may be automatically acquired. Also, workflow guidance for a patient may be provided based on analysis of the images of selected features.

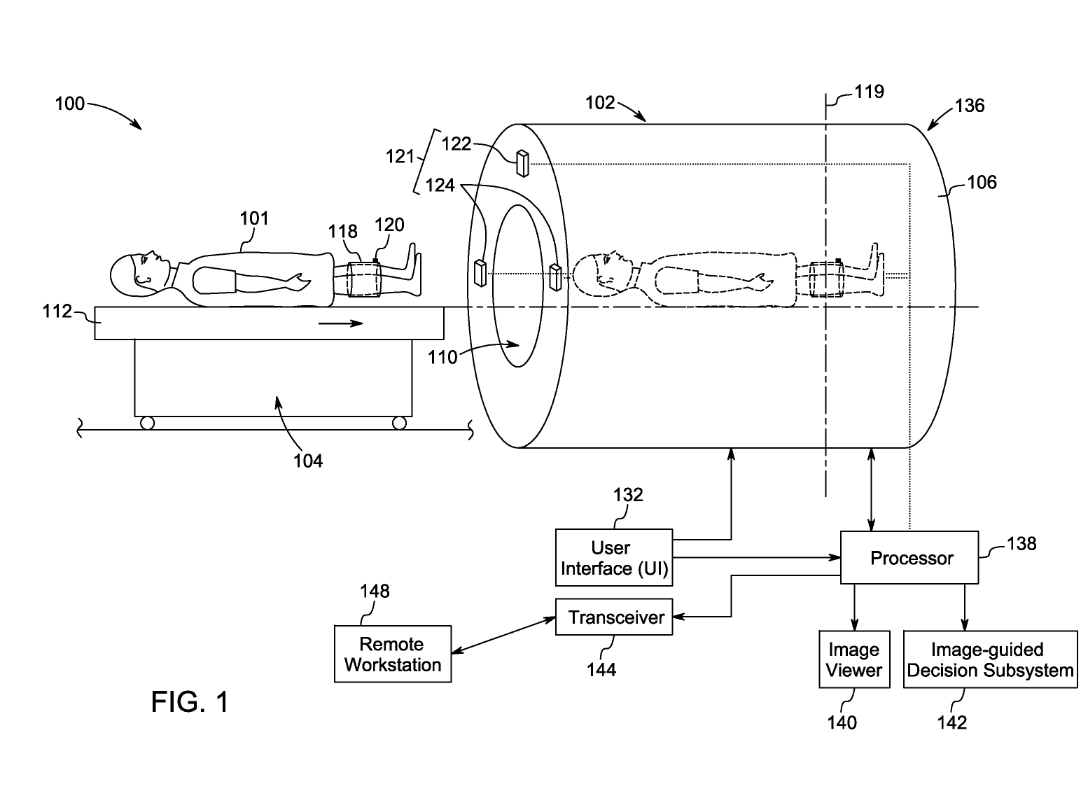

Although exemplary embodiments of the present technique are described in the context of a magnetic resonance imaging (MRI) operation, it will be appreciated that use of the present technique in various other imaging applications and systems is also contemplated. Some of these systems, for example, may include CT imaging systems, PET imaging systems, optical imaging systems, and hybrid systems combining MR with other modalities. An exemplary environment that is suitable for practicing various implementations of the present technique is discussed in the following sections with reference to FIG. 1.

FIG. 1 illustrates an exemplary system 100 for use in automatic acquisition of optimized images of a subject, such as a patient 101. The optimized images may be referred to as images that are of sufficient quality and aids in making effective and accurate clinical diagnosis. Further, it may be noted that the terms “patient” and “subject” may be used interchangeably in the below description. For discussion purposes, the system 100 is described with reference to patient preparation in an MR imaging operation. Accordingly, in one embodiment, the system 100 includes a user interface 132, a scanning unit 136, a processor 138, an image viewer 140, an image-guided decision subsystem 142, a transceiver 144, and a remote workstation 148. The user interface 132 may be operatively coupled to the scanning unit 136 and the processor 138. Also, the user interface 132 may be used to provide one or more inputs to the scanning unit 136 and/or the processor 138.

In a presently contemplated configuration, as the patient arrives for scanning, the operator may employ the user interface 132 to specify an anatomical region of the patient to be scanned. In one example, the anatomical region may be an abdomen portion of the subject 101.

Additionally, the operator may use the user interface 132 for selecting one or more features of interest and one or more appropriate properties of the features of interest that will be imaged. In one example, the one or more features of interest may include organs, tumors, and/or other lesions in the anatomical region of the subject 101. In another example, the one or more appropriate or necessary properties of the features of interest may include image contrast, signal-to-noise ratio (SNR), contrast-to-noise ratio (CNR), blood flow, computed parametric maps, and others.

In one embodiment, the operator may select predetermined scan parameters that are associated with the features of interest of the examination. In one embodiment, the predetermined scan parameters may be selected from a pre-determined protocol that is deemed as the most suitable or appropriate for the features of interest. For example, the water-based tissues may have scan parameters that are different from scan parameters of the fat-based tissue. Thus, the operator may select the scan parameters that are associated with the desired feature of interest in the anatomical region. Additionally, other higher level examination characteristics or properties such as “brain tumor” or “stroke” may be selected, possibly selecting multiple imaging acquisitions with differing scan parameters, together with multiple image processing or computational options. In the case of “stroke”, this could be fractional anisotropy or apparent diffusion coefficient together with a brain perfusion acquisition. Also, the knowledge of the scan parameters to select for a specific feature is embedded in the processor 138, and may be implemented as scan protocols.

In another embodiment, the operator may select the high level examination characteristic or property such as “rule out tumor”, and the study feature may include the requirement to scan multiple regions of the body. In this example, the operator either selects the anatomic region to be scanned or have the processor 138 determine what anatomic region or organs are in the current imaging field-of-view. Applying knowledge of what acquisition parameters and image feature characteristics and properties are appropriate and ideal to meet the requirements of the high level examination characteristic or property, the appropriate scan acquisition parameters are implemented as scan protocol for that region of the body. A specific example in an imaging modality other than magnetic resonance imaging (MRI) is in computed tomography (CT) where the kV and mA settings may change depending on the region of the body to provide desired image contrast but maintain minimum X-ray dose to the patient.

After selecting one or more features of interest and one or more appropriate properties of the features of interest, the scanning unit 136 may be configured to scan the anatomical region that is specified by the operator. As depicted in FIG. 1, the scanning unit 136 may be operatively coupled to the user interface 132 and the processor 138. The scanning unit 136 may include a magnetostatic field generator 102 operatively coupled to a motorized table unit 104. Further, the magnetostatic field generator 102 includes a magnet 106, for example, including RF or gradient coils and a bore 110 to accommodate the patient 101, in one implementation, disposed in a supine position. In certain other implementations, however, the patient 101 may be disposed in other positions suitable for imaging. In one example, the table unit 104 includes a positioning unit (not shown) that governs motion of the patient cradle 112, and thus, the patient position within the magnet 106.

Once the patient or subject 101 is positioned on the table, the scanning unit 136 moves the patient to isocenter of a magnet 106 in the scanning unit 136. The isocenter of the magnet 106 has good magnetic field homogeneity and good linearity of the gradient fields, and thus, it is desirable to position the anatomical region at the isocenter of the magnet. The scanning unit 136 further starts to scan the patient 101 based on a standard imaging protocol. In one example, the scanning unit 136 automatically scans the patient 101 using perhaps a single button push on the UI. The section of the anatomy that is scanned is usually determined by how the scan operator positions the patient 101 on the table and which location is set as the patient landmark. This may or may not be the correct part of the anatomy that is to be scanned. Scanning or image acquisition commences, with the first acquired images used to ensure that the correct part of the anatomy is being scanned. This is accomplished by using the processor 138 to identify anatomical landmarks (e.g. lungs, kidneys, or liver) that would indicate the section of the body that is scanned.

Furthermore, the processor 138 may be configured to automatically process one or more medical images acquired by the scanning unit 136. Particularly, the processor 138 may be configured to algorithmically process the acquired medical images in real-time to identify a feature of interest, recording feature position, size, and other properties. In one example, the feature may be recognized in three dimensions. In one embodiment, the processor 138 may identify a boundary of the feature of interest that is selected by the operator 138.

In an alternative embodiment, the operator may use a survey scan of the anatomical region for manually identifying a feature of interest. Particularly, the operator may interact with the survey scan and may select the feature of interest in the anatomical region of the patient 101. Further, the operator may instruct the processor 138 to run one or more algorithms over the selected feature to determine a boundary of the feature. In one embodiment, the operator may trace along the outline of the feature of interest.

Upon identifying the features, the processor 138 may automatically instruct the scanning unit 136 to re-scan the identified feature of interest using properties selected or pre-selected by the operator. In one example, the properties may include image signal-to-noise ratio (SNR), contrast-to-noise ratio (CNR), blood flow, or other features configured by the clinician or clinical site. These properties may have characteristics that ensure that the images are of sufficient quality to enable effective and accurate clinical diagnosis. These images may be referred as optimized images. Particularly, the scanning unit 136 may re-scan the identified feature of interest using the one or more selected properties for automatically acquiring the one or more optimized images of the identified feature of interest. In one example, the processor 138 may instruct the scanning unit to acquire an image of sufficiently high CNR of the feature of interest. In another example, the processor 138 may instruct the scanning unit 136 to acquire an image with sufficiently high SNR of the feature of interest. Other image feature characteristics or properties to be studied such as “brain tumor” may also be selected to enhance the ability to visualize a disease in the anatomical region of the patient 101. In one embodiment, if multiple image characteristics or properties are selected to provide sufficient information for effective and accurate clinical diagnosis, the feature of interest may be imaged multiple times at the same slice locations to fulfill the request. It may be noted that image acquisition and feature recognition are performed as part of an automated workflow.

In one embodiment, characteristics or properties of the images may be compared with the characteristics or properties of previous images or pre-stored images to determine changes in the anatomical region of the patient. This in turn may aid in determining diagnosing changes that is used for monitoring the effectiveness of therapy or monitoring disease progression. For example, if a patient has been prescribed a regimen of chemotherapy, an oncologist would like to determine if the particular combination of treatment drugs is effective as early as possible in the therapy treatment cycle. This allows the oncologist to modify or even change the chemotherapy components. These changes can be determined using imaging, such as MRI. One method to observe or measure these changes is to acquire images of the specific anatomical region with the same image acquisition parameters (imaging protocol) and also of the same image orientation in the follow-up examination.

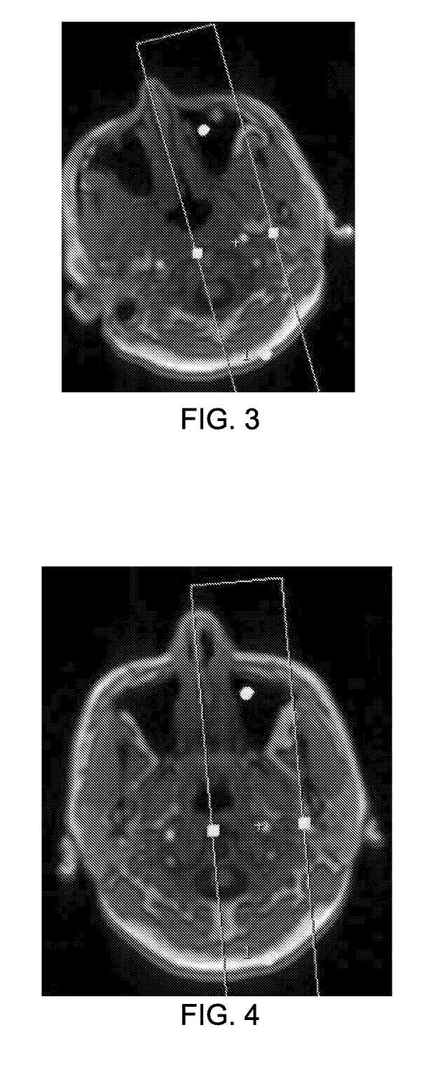

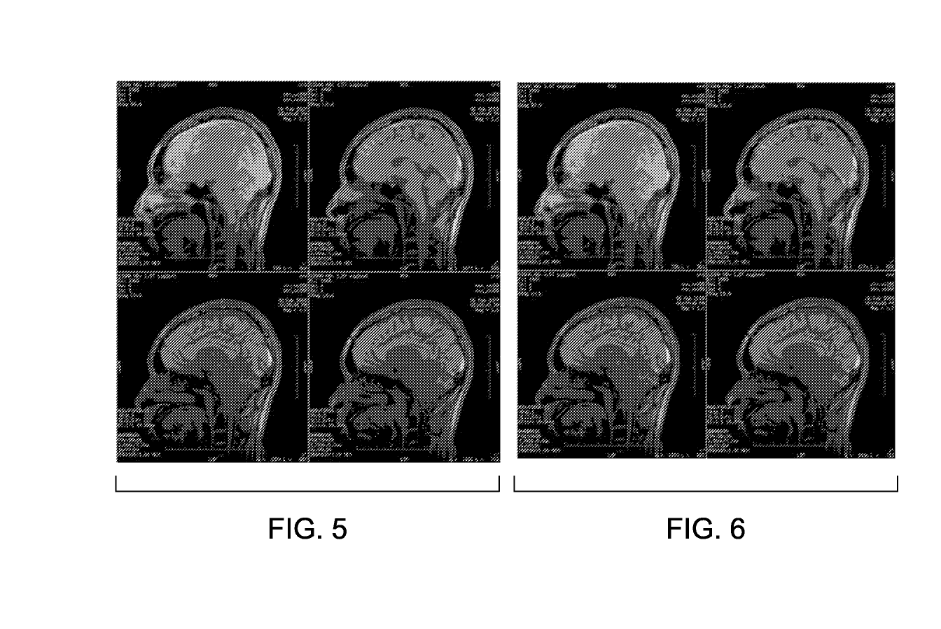

In one embodiment of a follow-up scan, the processor 138 may analyze the first acquired images to determine the anatomy to be studied. Also, anatomical landmarks in the anatomy that are identified by using suitable algorithms may be used for determining an ideal anatomy. Further, images acquired from the ideal anatomy may be used in the follow-up examination to compare with images acquired in a prior examination. In one example, a processor 138 may acquire an image of the brain, as depicted in FIG. 3, where the desired characteristic or appropriate property is needed to monitor changes in the pituitary gland. Subsequently, in a later patient encounter or imaging examination, the processor 138 may interrogate the acquired image to determine the ideal image acquisition plane for a follow-up study. The ideal image acquisition plane is depicted as an outlined box 402 superimposed on the image, as shown in FIG. 4. Thereafter, the processor 138 may determine the correct image acquisition plane, as depicted in FIG. 5. Further, the correct image acquisition plane matches an earlier acquired image of the same anatomy. The earlier acquired image of the same anatomy is shown in FIG. 6. In addition, the processor 138 determines other image acquisition parameters such that the subsequent images have the desired image contrast, SNR, and CNR or other specified characteristics for a follow-up examination. In this example, these images show the pituitary gland with the desired or required image contrast. Having the same image scan plane and image characteristics and features as that of an earlier imaging examination helps the clinician quickly identify changes that would be indicative of disease progression or therapy effectiveness or remission.

In one embodiment, the operator may use an interactive session to select one or more desired image characteristics or appropriate properties that were not selected prior to initiating the scan. Further, the processor 138 may instruct the scanning unit to re-acquire images with the selected desired image characteristics or properties. Additionally, the system 100 may permit the operator to view acquired images from the anatomical region while interactively adjusting specific desired characteristics or properties of the images. Particularly, during an interactive session, the operator reviews images with the image viewer 140 as is currently done on MRI systems, but with an additional capability. For an image or image set, the operator uses the image viewer control to select the desired image characteristic or image parameter of interest, e.g., image contrast. This enables the previously acquired image-contrast images to be displayed. If a desired image characteristic or property/parameter is selected that has not been imaged, the scanning unit 136 may scan the feature of interest, optimizing the acquisition using the selected property, and make the images available for operator viewing. If done during a post-processing session, the operator is restricted to optimized properties that were previously selected for that study, and to the images that have already been acquired. This results in perhaps sub-optimal post-processed or computed parametric images as the ideal post-processed images may require additional images for computation.

If during an interactive session, specific post-processed images, such as computed parametric maps are desired, the processor 138 may determine that sufficient images necessary to compute the parametric maps with sufficient accuracy are absent. In this case, the processor 138 may suggest additional images to the operator that need to be acquired or automatically proceed to acquire the necessary additional images. It may be noted that the parametric maps or parametric images are referred to as images that are derived from computational or analysis algorithms that utilize at least two previously acquired images. An example of parametric images is computed maps of T₂* values that are generated from fitting pixel values from a series of gradient-recalled echo MRI images, each acquired at a different echo time (TE) according to the below mentioned equation:

S(TE)=Sₒexp(−T₂*/TE) (1)

Where T₂* is the transverse relaxation time that is dependent on spin-spin interactions and magnetic field inhomogeneities, TE is the echo time, and Sₒ is the equilibrium signal amplitude. Other examples of parameteric images may include maps of apparent diffusion coefficient (ADC) or diffusion anisotropy that are computed from a series of MRI images that have different diffusion weighting along different directions. Parametric images or parametric maps may provide another measure of information in the form of images that are derived from a specific series of acquired MR images acquired in a specific manner or using a specific prescribed image acquisition protocol.

Furthermore, during an interactive session, the image viewer 140 may also display computed parametric images based on the pre-determined image acquisition protocol that is determined by the study type and anatomical section previously identified. This provides further images for the operator/clinician to view and more rapidly make an accurate clinical assessment. These images are then determined as optimized as they would have sufficient image properties and quality that effective and accurate clinical diagnosis may be made. In one embodiment, the optimized images may be determined based one or more factors, such as imaging correct anatomy, ensuring that the entire organ or section of the anatomy is included in the acquired images, correct orientation of images such that they may be compared to a prior imaging study so that anatomical or physiological changes can be better interpreted, or the correct imaging protocol is used to match that for a specific region of the patient.

Moreover, the image viewer 140 also permits viewing of multiple optimally acquired or processed images that can be displayed simultaneously, such as blood flow and image contrast type, such as T₁-weighted images or T₂-weighted images, for example, by displaying one set of images as a semi-transparent overlay over a second set of images. The amount of transparency is controllable by the operator. If viewing a feature with multiple parameters selected, multiple viewing windows are also available to the operator.

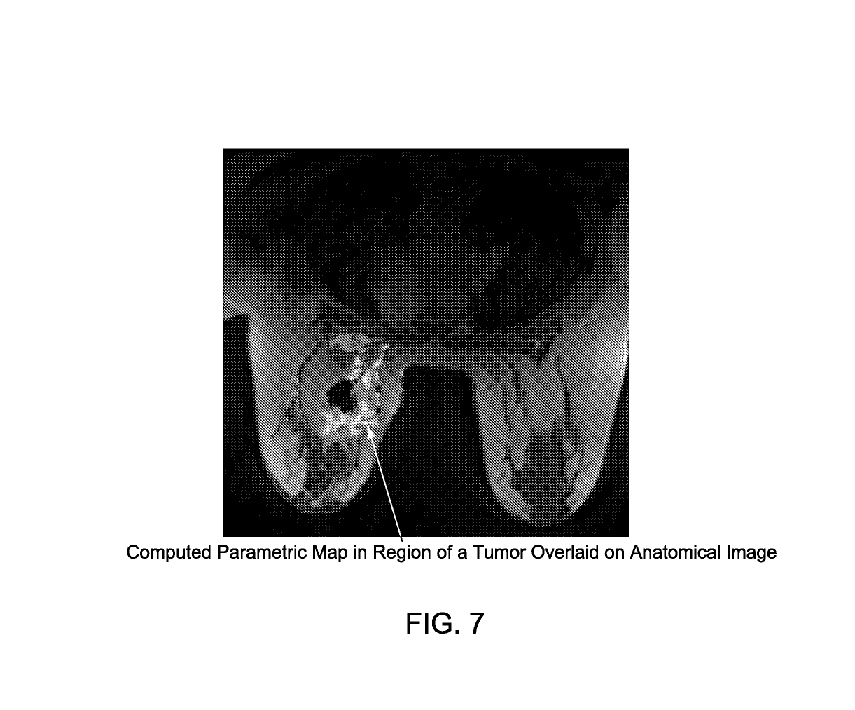

Upon obtaining the optimized images, the processor 138 may send these optimized images to the image-guided decision subsystem 142 for further image analysis of the feature of interest and for providing guidance to the clinician. Such analysis may be algorithms-based image processing to compute a specific property or characteristic of the anatomy that is the subject of the clinical examination. An example is shown in FIG. 7, where a computed parametric map of tissue permeability (K^{trans}) is overlaid as a color map (shown with an arrow in FIG. 7) over a T₁-weighted image for a breast MRI study of a patient with breast cancer. Particularly, the image-guided decision subsystem 142 may process these optimized images using one or more algorithms to provide image-guided decision support or clinical information to the clinician. In one example, the image-guided decision support or clinical information includes characteristics of a disease state that are manifested in particular image contrast or image properties or other image metrics specific to a particular disease state. This information may be accumulated from a collection of studies performed on a number of different patients with known outcomes and stored in a database that is accessible by the image-guided decision subsystem 142. Metrics include morphologic features such as shape, speculation and heterogeneity, and physiologic features such contrast uptake or model-based perfusion. Examples of computed parametric image properties that may be utilized are tissue T₁ relaxation, T₂ or T₂* relaxation, fractional anisotropy, etc. The computed parametric images are the result of a prior image acquisition such as a multi-echo TE acquisition for generating T₂ or T₂* maps or a diffusion tensor image acquisition for generating fractional anisotropy or apparent diffusion coefficient maps.

In addition, the image-guided decision subsystem 142 may compute feature differences with prior imaging studies of the same patient. In one example, the image-guided decision subsystem 142 may determine a difference between the current acquired images of the at least one feature of interest and images of the same patient of the same feature of interest that were acquired in a prior examination. In addition, the image-guided decision subsystem 142 may consult a database to identify prior cases with similar characteristics as the current study, providing information allowing the clinician to make a more informed and accurate diagnosis.

One application of this capability is in therapy monitoring. There are also feature-specific computer aided diagnostic tools to operate on images of a feature of interest. If the image-guided decision subsystem 142 determines additional images are required for processing, it will perform the acquisition through the scanning unit 136. Further, if the images are already acquired and the system 100 is being operated in a post-processing mode, the image-guided decision subsystem 142 may remember the requirements until the patient 101 is imaged for a follow-up examination at a later time. Moreover, the image-guided decision subsystem 142 may also make use of remote servers for rapid processing of data. After processing of the mages, the image-guided decision subsystem 142 may create a report with guidance on selected features, and with recommendations and clinical information for further patient workflow.

Alternatively, the processor 138 may communicate the acquired or processed optimized images of a feature of interest to the remote workstation 148 via the transceiver 144. Also, these acquired or processed optimized images of a feature of interest may be viewed on the remote workstation 148. In one example, the remote workstation 148 may be referred to as a patient archival and communications (PACS) workstation that is communicatively coupled to the transceiver 144.

In one embodiment, control of the interactive scanning sessions may be accomplished from the PACS workstation 148 by a radiologist. Particularly, a technologist may be present at the scanner for patient safety and setup. Further, the technologist may perform the study and then may allow the radiologist to take control of the session to view images, and determine if additional images are required. While the radiologist is controlling the system 100, the technologist is still able to view the user interface (UI) 132 and the image viewer 140 as it is manipulated by the radiologist. In addition, the technologist and radiologist may remain in contact using an audio feature of the system 100. In this scenario, the radiologist's involvement is primarily viewing, but with the patient 101 still in the scanning unit 136, permits additional image acquisition if required. This eliminates future sessions to acquire or reacquire image data. This capability is aligned with emerging trends placing additional capability on PACS workstation 148 and integration of PACS workstations 148 with hospital systems.

Thus, an automated approach permits a less skilled operator to reproducibly produce images optimized for specific properties, and does not require intimate knowledge of patient anatomy or of the scan protocols used to produce these optimized images. This system also permits novel composite images with all features of interest optimized to ensure that the acquired or processed images are of sufficient quality and have sufficient information content to allow effective and accurate clinical diagnosis.

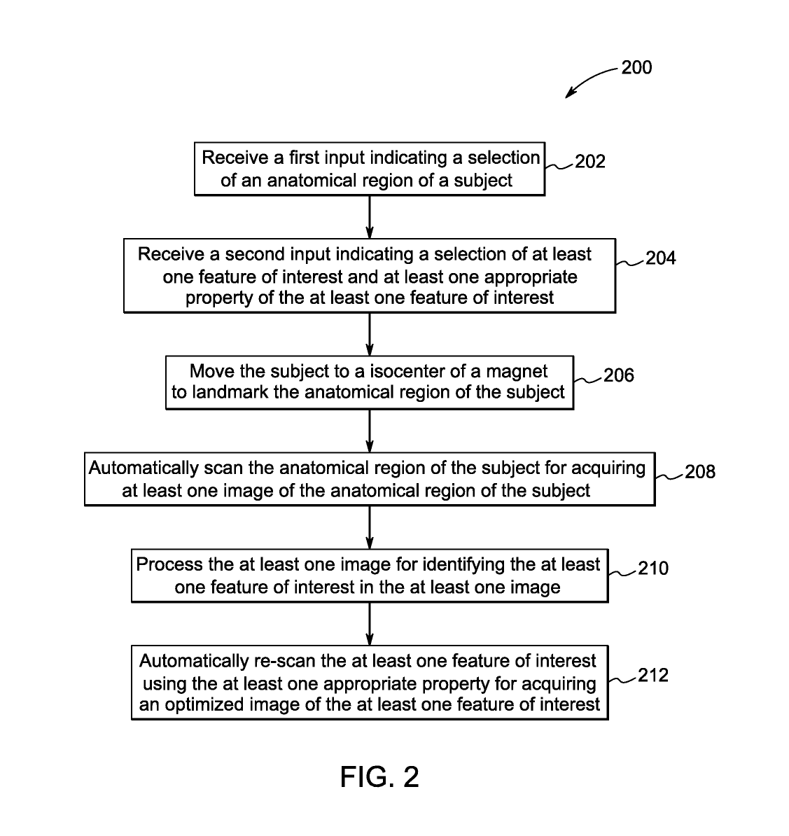

Referring to FIG. 2, a flowchart depicting an exemplary method for acquiring an optimized image of an object, in accordance with aspects of the present disclosure, is depicted. For ease of understanding, the method 200 is described with reference to the components of FIG. 1. The method 200 begins with step 202, where a first input indicating a selection of an anatomical region of a subject 101 is received. To that end, a user interface 132 is configured to receive the first input from an operator/clinician. Particularly, as the patient arrives for scanning, the operator may employ the user interface 132 to specify the anatomical region of the subject 101 to be scanned. In one example, the anatomical region may be an abdomen portion of the subject 101.

Subsequently, at step 204, a second input indicating a selection of at least one feature of interest and at least one appropriate property of the at least one feature of interest is received from the operator. Particularly, the operator may use the user interface 132 for selecting one or more features of interest and one or more appropriate properties of the features of interest. In one example, the one or more features of interest may include organs, tumors, and/or other lesions in the anatomical region of the subject 101. In another example, the one or more appropriate properties of the features of interest may include image contrast, SNR, CNR, blood flow and others that are deemed necessary to provide image data to ensure effective and accurate clinical diagnosis

Further, at step 206, the subject 101 is moved to the isocenter of a magnet 106 in a magneto static field generator 102. In one example, the isocenter of the magnet 106 is a region of the magnetostatic field generator 102 where the magnetic field is homogeneous and the gradient fields are substantially linear. Thus, the isocenter region is considered as the ideal region for conducting MRI studies. Also, MRI operators are trained to ensure that the region of the anatomy to be imaged is substantially within the isocenter region of the magnet 106. In one example, the MRI scan operator may select the approximate region of the anatomy to be examined by setting a landmark on the subject or patient 101 using the scanning unit 136.

Additionally, at step 208, the anatomical region of the subject 101 may be automatically scanned for acquiring at least one image of the anatomical region of the subject 101. Particularly, the scanning unit 136 may scan the anatomical region to acquire one or more images of the anatomical region using one or more imaging protocols.

At step 210, the at least one image is processed for identifying the at least one feature of interest in the at least one image. To that end, the processor 138 may be configured to process one or more medical images acquired by the scanning unit 136. Particularly, the processor 138 may be configured to algorithmically process the acquired medical images in real-time to identify a feature of interest, recording feature position, size, and other properties.

Subsequently, at step 212, the at least one feature of interest is automatically re-scanned using the at least one selected property for acquiring an image with optimized characteristics or properties of the at least one feature of interest. In one embodiment, this image may be referred as an optimized image. To that end, the processor 138 may instruct the scanning unit 136 to re-scan the identified feature of interest using one or more image characteristics or appropriate properties that are selected by the operator. In one embodiment, if multiple image characteristics or properties are selected, the feature of interest may be imaged multiple times at the same slice locations to fulfill the request. It may be noted that image acquisition and feature recognition are performed as part of an automated workflow. Such optimizations could also include re-scanning the patient 101 to ensure that the appropriate or ideal scan planes are used for the examination. These are determined by the processor 138 from processing the initial or first images.

In one embodiment, the scanning unit 136 may be a computed tomography (CT) unit. Also, the selected appropriate property may include one or more parameters that are used to improve quality of the optimized image, while reducing dose or maintaining low dose on the anatomical region of the subject. Also, the CT unit with the one or more selected parameters may be used for automatic identification of the anatomical region of the patient and then automatically modifying the acquisition protocol to provide good image quality of the optimized image. In this example, the anatomical region of the patient may include organ or section of the patient/body.

Upon obtaining the optimized images of the feature of interest, the processor 138 may send these optimized images to the image-guided decision subsystem 142 for analysis of the feature of interest and for providing guidance to the clinician. In one example, the image-guided decision support includes parameterization or computation of different physiologic properties of the anatomy of interest as parametric maps to determine metrics specific to a disease. Metrics include morphologic features such as shape, speculation and heterogeneity, and physiologic features such contrast uptake or model-based perfusion, as depicted in FIG. 7.

Alternatively, the processor 138 may also compute and display parameterized images in real-time so that information is presented to the clinician as well as the other images. Furthermore, the computed parameterized images may be sent to the image-guided decision subsystem 142 for analysis and providing subsequent informed guidance.

Alternatively, the processor 138 may communicate the optimized images of the feature of interest to a remote workstation 148, such as PACS via a transceiver 144 for analysis of the feature of interest. In this scenario, the radiologist may be involved in primary viewing of the images, but with the subject 101 still in the scanning unit 136. This procedure may permit additional image acquisition if required. Also, this eliminates future sessions to acquire or reacquire image data.

The various embodiments of the system and the method may be used for performing feature-specific analysis and producing metrics characterizing the features. Also, the system and the method may produce guidance for the clinician relative to feature related issues. This is also of importance in value systems where the skill of the technologist/radiographer is low or inexperienced.

Although specific features of various embodiments of the invention may be shown in and/or described with respect to some drawings and not in others, this is for convenience only. It is to be understood that the described features, structures, and/or characteristics may be combined and/or used interchangeably in any suitable manner in the various embodiments, for example, to construct additional assemblies and techniques. Further, while only certain features of the present invention have been illustrated and described herein, many modifications and changes will occur to those skilled in the art. It is, therefore, to be understood that the appended claims are intended to cover all such modifications and changes as fall within the true spirit of the invention.

1. A method comprising:
receiving a first input indicating a selection of an anatomical region of a subject;
receiving a second input indicating a selection of at least one feature of interest and at least one appropriate property of the at least one feature of interest;
moving the subject to isocenter of a magnet;
automatically scanning the anatomical region of the subject for acquiring at least one image of the anatomical region of the subject;
processing the at least one image for identifying the at least one feature of interest in the at least one image; and
automatically re-scanning the at least one feature of interest using the at least one appropriate property for acquiring an optimized image of the at least one feature of interest.

2. The method of claim 1, further comprising displaying the optimized image of the at least one feature of interest.

3. The method of claim 1, further comprising automatically communicating the optimized image of the at least one feature of interest to a remote workstation.

4. The method of claim 1, further comprising automatically processing the optimized image of the at least one feature of interest to determine clinical information associated with the at least one feature of interest.

5. The method of claim 4, wherein the clinical information comprises metrics associated with the at least one feature of interest.

6. The method of claim 1, further comprising automatically processing the at least one acquired image to generate parametric maps of tissue properties and to determine clinical information associated with at least one feature of interest.

7. The method of claim 6, wherein the clinical information comprises computed parametric images associated with at least one feature of interest.

8. The method of claim 7, wherein computed parametric images include tissue information of at least one feature of interest that is indicative of disease or normality.

9. The method of claim 4, wherein automatically processing the optimized image comprises determining a difference between the optimized image and one or more pre-stored images of the at least one feature of interest.

10. The method of claim 1, wherein processing the at least one image comprises automatically processing the at least one image in a real time for identifying at least one of a position, a size, and a boundary of the at least one feature of interest.

11. The method of claim 1, wherein processing the at least one image comprises survey scanning the at least one image for manually identifying the at least one feature of interest.

12. The method of claim 1, wherein the at least one appropriate property of the at least one feature of interest is received after identifying the at least one feature of interest in the at least one image.

13. The method of claim 1, wherein the at least one appropriate property of the at least one feature of interest is adjusted or changed prior to re-scanning the at least one feature of interest.

14. A system comprising:
an user interface configured to:
receive a first input indicating a selection of an anatomical region of a subject;
receive a second input indicating a selection of at least one feature of interest and at least one appropriate property of the at least one feature of interest;
a scanning unit coupled to the user interface and configured to move the subject to isocenter of a magnet to landmark the anatomical region of the subject and acquiring at least one image of the anatomical region of the subject; and
a processor coupled to the scanning unit and configured to process the at least one image for identifying the at least one feature of interest in the at least one image,
wherein the scanning unit is configured to re-scan the at least one feature of interest using the at least one selected appropriate property for acquiring an optimized image of the at least one feature of interest.

15. The system of claim 14, further comprising a display unit configured to display the optimized image of the at least one feature of interest.

16. The system of claim 14, further comprising a transceiver configured to automatically communicate the optimized image of the at least one feature of interest to a remote workstation.

17. The system of claim 14, further comprising an image-guided decision subsystem operatively coupled to the processor and configured to automatically process the optimized image of the at least one feature of interest to determine clinical information associated with the at least one feature of interest.

18. The system of claim 17, wherein the clinical information comprises metrics associated to the at least one feature of interest image.

19. The system of claim 17, wherein the clinical information comprises metrics associated with at least one feature of interest from the optimized image compared to features of interest of pre-stored images.

20. The system of claim 17, wherein the image-guided decision subsystem is configured to automatically process the optimized image for determining a difference between the at least one feature of interest image and a pre-stored feature of interest image.

21. The system of claim 14, wherein the processor is configured to automatically process the at least one image in real-time for identifying at least one of a position, a size, and a boundary of the at least one feature of interest.

22. The system of claim 14, wherein the at least one feature of interest is manually identified by survey scanning the at least one image.

23. The system of claim 14, wherein the at least one appropriate property of the at least one feature of interest is received after identifying the at least one feature of interest in the at least one image.

24. The system of claim 14, wherein the at least one appropriate property of the at least one feature of interest is adjusted or changed prior to re-scanning the at least one feature of interest.

25. The system of claim 14, further comprising a transceiver operatively coupled to the processor and configured to communicate the optimized image of the at least one feature of interest to a remote workstation.

26. The system of claim 25, wherein the remote workstation is configured to control the scanning unit based on the received optimized image of the at least one feature of interest.

27. The system of claim 14, wherein the scanning unit includes a computed tomography (CT) unit, wherein the appropriate property includes one or more parameters that are used to improve quality of the optimized image while reducing dose or maintaining low dose on the anatomical region of the subject.
